(19) United States
(12) Patent Application Publication
(10) Pub. No.: US 2012/0065538 A1
(43) Pub. Date: Mar. 15, 2012

(54) ELECTROMYOGRAPHIC (EMG) DEVICE FOR THE DIAGNOSIS AND TREATMENT OF MUSCLE INJURIES

(75) Inventor: Evan B. FRIEDMAN, Vaughan (CA)

(73) Assignee: INTRONIX TECHNOLOGIES CORPORATION, Bolton (CA)

(21) Appl. No.: 13/232,153

(22) Filed: Sep. 14, 2011

Related U.S. Application Data
(60) Provisional application No. 61/383,202, filed on Sep. 15, 2010.

(51) Int. Cl. A61B 5/0488

(52) U.S. Cl. 600/546

(57) ABSTRACT

An electromyographic (EMG) device for the diagnosis and treatment of muscle injuries associated with pain, stiffness, and movement disorders which is configured for diagnosing chronic and recurrent muscle pain and which includes two active EMG electrodes and an EMG reference electrode which when attached to the subject patient can measure and display electromyographic signals. The device includes an integrated stimulator that can be used for treating chronic and recurrent muscle pain, by evoking stimulation, or E-stim, location. The device includes a visual display system for displaying raw and processed EMG and is configured to provide for audio feedback based on the raw EMG.

### CROSS-REFERENCE TO RELATED APPLICATION

This application claims priority to U.S. Provisional Application No. 61/383,202, filed on Sep. 15, 2010, the entire contents of which are incorporated herein by reference.

### FIELD OF THE INVENTION

The present invention relates to an electromyographic (EMG) device for the diagnosis and treatment of muscle injuries associated with pain, stiffness, and movement disorders.

### BACKGROUND OF THE INVENTION

It is estimated that up to 20% of the adult population in North America suffers from chronic and recurrent muscle pain. Over 240 million, or 80% of the North American population will develop backache during their lives. In 2005, North Americans spent in excess of $85.9 billion looking for relief from back and neck pain, up 65% from $52.1 billion in 1997. About 25 million people in North America suffer from movement disorders, which range from being uncomfortable, to debilitating chronic and recurring muscle pain, and spasticity, are neuromuscular abnormalities typically following a strain injury or physical damage, and manifested by what has been termed trigger point (TrP) phenomena. As used herein, trigger point means a localized area of tenderness within a muscle, and is usually associated with spontaneous electromyographic (EMG) activity. Thus, a trigger point is a location of spontaneous EMG activity within a muscle associated with pain. The trigger point locations are found by physical examination, and manual palpation. They are found to be nodular in nature, or “tight spots” in the muscle.

Trigger points may be within muscle spindles. TrPs can be objectively diagnosed by identification of spontaneous EMG activity in a trigger point while adjacent muscle fibers are electromyographically quiet. Once the trigger point EMG activity is identified, chronic and recurrent muscle pain associated with this localized EMG activity can be treated.

Treatment can involve insertion of regular, or hypodermic, needle electrodes to deliver treatment. Treatment can include insertion of a dry needle, or use of medications including sympathetic blocking agents, saline (including cryogenic Tx), local anaesthetic, botulinum toxin variants, and phenols (and other neurolytics).

Medical clinics that diagnose and treat movement disorders and muscular pain use a variety of devices as components to their patient care regimen. Unfortunately, the availability of effective devices is limited which adversely affects the quality of treatment. Needle EMG guidance offers a simple and effective methodology for locating sites of muscular spasm and associated pain. It is shown, and supported, in the literature to: increase treatment efficacy; assist with the identification of involved muscles i.e. pre-injection physio-pathological evaluation or pre-intervention evaluation’; confirm the location of the affected muscles that are too deep to be visualized and may be surrounded by essential nerves and blood vessels; provide confirmation of treatment effects; and increase targeting accuracy and maximizes efficiency with reduced neuromodulator drug dose, thereby reducing the incidence of drug resistance, and limiting drug diffusion into adjacent areas; be an important tool for new injectors to confirm injection points.

Clinicians using similar techniques are forced to use EMG amplifiers and stimulators, from different manufacturers, to assemble their own systems. This requires a degree of technical skill and access to the right equipment. Most attempts lead to basic tools with little performance other than EMG signal monitoring and possibly, stimulation for muscle twitch. Often, clinicians rely on simple palpation of trigger points (knots) to identify injection targets. Palpitation provides little information on the depth of injection and proximity to adjacent vital tissue. Often times increased volume of drug is required to account for lack of accuracy. This can lead to drug resistance and increased side effects due to drug diffusion into adjacent areas.

The dedicated products currently available in the needle EMG guidance market are handheld, battery operated devices. They are capable of both EMG audio monitoring and may, or may not, include a stimulator which can be either current, or voltage, stimulation, typically ranging from 0-15 mA. The leading device is set up to use Medtronic electrodes only, which limits choice, its stimulators are weak (which can impact motivating larger muscles) and the EMG audio tone is typically poor. There are no displays for monitoring the EMG signal or status. There are no instantaneous indicators, such as integrated EMG or RMS to show overall EMG energy.

What is therefore needed is an electromyographic (EMG) device for the diagnosis and treatment of muscle injuries associated with pain, stiffness, and movement disorders which avoids the above-mentioned problems.

### SUMMARY OF THE INVENTION

The present invention provides a electromyographic (EMG) device for the diagnosis and treatment of muscle injuries associated with pain, stiffness, and movement disorders.

To these ends, in a first aspect of the invention, an electromyographic (EMG) device for diagnosing chronic and recurrent muscle pain preferably connects to two active EMG electrodes and an EMG reference electrode.

In a second aspect of the invention, an integrated stimulator can be used for treating chronic and recurrent muscle pain, by evoking stimulation, or E-stim, location.

In a third aspect of the invention, a system is provided for display of raw and processed EMG on an easy to read LCD display, allowing visual analysis of the level of spontaneous EMG activity in trigger points and adjacent muscle tissue.

In a fourth aspect of the invention, a system is provided for propagation of audio feedback based on the raw EMG. This provides an auditory analysis of the level of spontaneous EMG activity in trigger points and adjacent muscle tissue.

An embodiment of the present invention provides an electromyographic (EMG) device, comprising:

a) a central processor controller and a user interface, an audio processing circuit connected to at least one speaker, an array of electrodes having first ends configured to be affixed to the skin of a subject patient, an electrode interface, said array of electrodes having second ends connected to said electrode interface, at least one of said electrodes is a hypodermic needle electrode;

b) an acquisition subsystem for measuring and recording electromyographic (EMG) activity connected to the central processor controller, the acquisition subsystem including
i) electromyographic signal detection and processing circuits connected to said electrode interface and said central processor controller for measuring electromyographic signals picked up by said array of electrodes and simultaneously transmitting said electromyographic signals to said audio processing circuit and processing said electromyographic signals to put them in a selected format and transmitting the formatted signals to said central processor controller;

c) a stimulation subsystem connected to the central processor controller, the stimulation subsystem including
i) stimulation logic circuit to control parameters of stimulation signals applied to the subject patient, said parameters including timing of stimulation signal frequency, stimulation signal pulse width, and stimulation signal pulse amplitude, the stimulation logic circuit being connected to the central processor controller,
ii) an electrical signal generator circuit connected to said central processor controller,
iii) a bridge output circuit connected to said stimulation logic circuit and said electrical signal generator circuit configured to produce said stimulation signals for application to the subject patient based on input from the stimulation logic circuit and the electrical signal generator circuit, the bridge circuit being connected to said electrode interface for delivering said stimulation signals to the subject patient through the array of electrodes;

d) a switch for switching between said acquisition subsystem and said stimulation subsystem; and

e) a visual display connect to said central processor controller configured to display to a clinician at least real-time electromyographic waveforms from said acquisition subsystem and stimulation parameters from said stimulation subsystem.

Other objects and advantages will appear as well hereinafter.

A further understanding of the functional and advantageous aspects of the invention can be realized by reference to the following detailed description and drawings.

### BRIEF DESCRIPTION OF THE DRAWINGS

The embodiments of the present invention are described with reference to the attached figures and table, wherein:

FIG. 1 shows a block diagram of a electromyographic (EMG) needle-injection device constructed in accordance with the present invention;

FIG. 2 shows EMG recording and stimulating electrodes;

FIG. 3 shows the EMG device flow diagram;

FIG. 4 is a perspective view of an embodiment of the electromyographic (EMG) device constructed in accordance with the present invention showing the main front panel of the packaged device; and

FIG. 5 is a perspective view of a bottom side of the electromyographic device.

### DETAILED DESCRIPTION OF THE INVENTION

Generally speaking, the systems described herein are directed to an electromyographic (EMG) device for the diagnosis and treatment of muscle injuries associated with pain, stiffness, and movement disorders. As required, embodiments of the present invention are disclosed herein. However, the disclosed embodiments are merely exemplary, and it should be understood that the invention may be embodied in many various and alternative forms. The figures are not to scale and some features may be exaggerated or minimized to show details of particular elements while related elements may have been eliminated to prevent obscuring novel aspects.

Therefore, specific structural and functional details disclosed herein are not to be interpreted as limiting but merely as a basis for the claims and as a representative basis for teaching one skilled in the art to variously employ the present invention. For purposes of teaching and not limitation, the illustrated embodiments are directed to an electromyographic (EMG) device for the diagnosis and treatment of muscle injuries associated with pain, stiffness, and movement disorders.

As used herein, the term “about” or “approximately”, when used in conjunction with ranges of dimensions, temperatures or other physical properties or characteristics is meant to cover slight variations that may exist in the upper and lower limits of the ranges of dimensions as to not exclude embodiments where on average most of the dimensions are satisfied but where statistically dimensions may exist outside this region. For example, in embodiments of the present invention dimensions of components of an apparatus and method of measuring optical properties of water are given but it will be understood that these are non-limiting.

As used herein, the coordinating conjunction “and/or” is meant to be a selection between a logical disjunction and a logical conjunction of the adjacent words, phrases, or clauses. Specifically, the phrase “X and/or Y” is meant to be interpreted as “one or both of X and Y” wherein X and Y are any word, phrase, or clause.

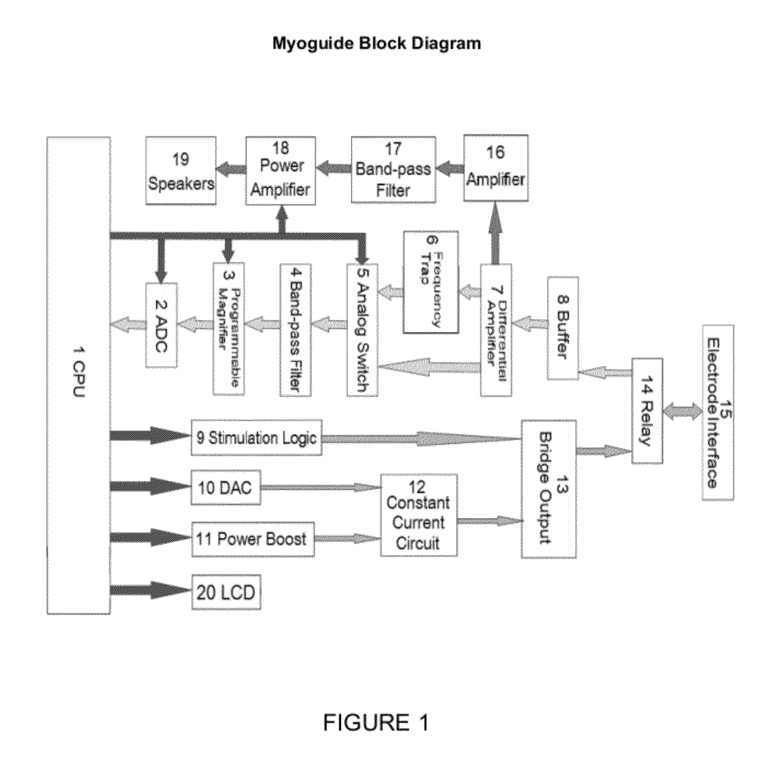

FIG. 1 shows a block diagram showing the components of the electromyographic (EMG) device. The device comprises two basic subsystems, a first being the acquisition subsystem and the other being a stimulation subsystem. The electromyographic (EMG) device is a battery powered, handheld, EMG amplifier with audio feedback, LCD EMG signal and device status display, and constant current stimulation ranging from 0-20 mA.

### Acquisition Subsystem

The electromyographic (EMG) device, including the acquisition subsystem and the stimulation subsystem are controlled by a central processor controller unit (CPU) 1 which is used for equipment waveform display, stimulus control, muscle tone volume control, and the like. An analog-to-digital converter (ADC) 2 connected to CPU 1 is used to convert processed EMG signals to digital signals. A programmable magnifier 3 connected to analog-to-digital converter 2 enables switching to achieve different levels of magnification. A band-pass filter 4 is used to filter out noise outside the EMG spectrum. An analog switch 5 is to toggle usage of a frequency trap circuit 6. Frequency trap 6 is used to filter out frequency noise. A differential amplifier 7 is used to amplify small EMG signals without distortion while satisfying the requirements for common-mode rejection ratio (CMRR) and short-circuit noise. Buffer 8 is used to increase input impedance. Differential amplifier 7 is connected to an amplifier 16 for performing further amplification for the EMG signals. A band-pass filter 17 connected to amplifier 16 is used to filter out noise outside the EMG spectrum on the signals output from amplifier 16. A power amplifier 18 which in one embodiment may be a class D audio amplifier, is used to drive the speakers 19 which in turn are used to convert electrical signals into audio signals.

As can be seen from FIG. 1, and mentioned above, the differential amplifier 7 is used to amplify small EMG signals and these signals are passed to the analog switch 5 to be further conditioned and transmitted to CPU 1 while at the same time being transmitted from differential amplifier 7 to amplifier 16 to be passed in turn to speakers 19. As described below in more detail, this allows simultaneous real-time visual display and audio processing.

### Stimulation Subsystem

A stimulation logic circuit 9 is used to control the timing of the stimulation circuit, which includes stimulation frequency, stimulation pulse width, etc. A digital-to-analog converter (DAC) 10 provides a variable reference voltage to the constant current circuit 12 discussed below, and hence achieve different stimulus current output. A power boost circuit 11 generates 300V of high voltage. A constant current generator circuit 12 connected to the digital-to-analog converter (DAC) 10 and the power boost circuit 11 helps the device in generating constant current stimulus output. A bridge output circuit 13 is connected to constant current generator circuit 12 is used to implement the stimulus output. A relay circuit 14 connected to bridge output circuit 13 and buffer circuit 8 is used to switch between the acquisition subsystem and the stimulation subsystem. An electrode interface 15 connected to the relay circuit 14 connects the electrode cables to the rest of the electromyographic (EMG) device.

A liquid crystal display (LCD) 20 is used, when the EMG device is used in EMG mode, to display real-time EMG waveforms, IEMG (Integrated EMG) histograms, latest RMS values and information of the currently displayed parameters. During operation of the EMG device in stimulation mode, LCD 20 is used to display the stimulation parameters such as current intensity, pulse width, frequency, and LCD backlight timing as well as the remaining battery power.

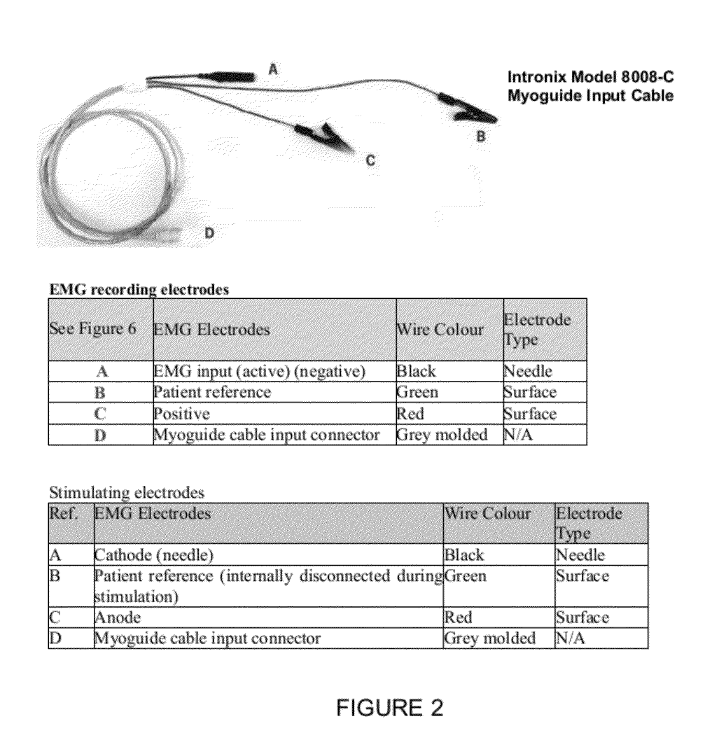

FIG. 2 shows the EMG recording electrodes in which electrode A is the EMG input active, negative electrode which is a needle. Electrode B is the patient reference surface electrode, and electrode C is positive surface electrode. Connector D is the EMG cable input connector connecting the electrodes A, B and C to electrode interface 15.

When in stimulation mode, the same electrodes are used, and electrode A (need) is the cathode, the patient reference electrode B is internally disconnected during stimulation, and electrode C is the anode.

During operation needle electrode A is inserted into the patient in the area of interest being studied, the electrode B is the patient reference surface electrode and is attached to a simple surface electrode connected to clip on the supplied input cable. The hook up is as simple as using a hypodermic needle electrode, which is the negative, to the black no touch connector. This is used to challenge the site of interest. The green connector, which can connect to either snap type or tab type surface electrodes, is used as a GROUND, and this is located in the vicinity, but no need to be overly close. The RED or positive is the reference used to compare against the hypodermic needle in a differential fashion. It is best located nearby the injection site. It is noted that these color schemes are following convention and are in no way considered limiting.

In operation the cable input connector D is connected to the EMG device housing. The other end of the cable has two colour keyed alligator clips and one touchproof male connector. The alligator clips can engage snap type, tab type or needle electrodes. The touchproof male connector is designed to engage the female connector of a hypodermic needle electrode (or conventional needle electrode, if dry needling). This connector can accommodate hypodermic needle electrodes from a wide variety of manufactures. The positive input runs out the red cable to an alligator clip. The negative input runs out the black cable as the inline touchproof connector. This connector is designed to engage the female mate from a (hypodermic) needle electrode. The reference input runs out the green cable to an alligator clip.

Once the electrode leads have been connected to the device, the ground and reference electrodes are attached to the patient, and when ready, one can proceed with the EMG needle electrode (active input). It is recommended to locate the reference electrode close to the investigational site, to avoid unnecessary noise and stimulation issues.

### Recording Mode

In recording mode the present EMG device is configured to amplify electrophysiological signals from muscle and provide audio feedback to assist clinicians in locating areas of muscle activity. The EMG device provides muscle and nerve localization information, to accurately guide and monitor needle electrode insertion, and/or injection of neuromodulator drugs, into a muscle in the human body. Any drug used will be that of the choice of the physician.

With reference to FIG. 1, in recording mode, the relay 14 is set to pass the input signal from the electrodes through the electrode interface 15 to the buffer 8. The EMG signal from the electrodes passes through the electrode interface 15 and through the relay 14 to the buffer 8. The buffer 8 serves to increase the input impedance before passing the signal to the differential amplifier 7. The differential amplifier 7 converts the differential signal to a single ended signal. The differential signal then passes the single ended input signal to the frequency trap 6, and the analog switch 5 for display processing and amplifier 16 for audio processing.

### Display Processing

In display processing, the frequency trap 6 removes the 60 Hz component of the signal to remove artifacts caused by 60 Hz interference. The analog switch 5 is controlled by the CPU 1 and serves to control whether the filtered signal from the frequency trap 6 or the raw signal from the differential amplifier 7 is passed to the bandpass filter 4 depending on the user adjustable notch filter parameter. The band-pass filter 4 serves to filter out the noise outside of the EMG spectrum. After band-pass filtering, the signal is passed to the programmable magnifier 3, controlled by the CPU 1, which amplifies the signal based on the user set vertical sensitivity parameter. The amplified analog signal is then converted to a digital signal and sent to the CPU 1 for further display processing. Once the digital signal is aquired by the CPU 1, the CPU 1 then determines the display speed based on the user adjustable horizontal sensitivity parameter, calculates the EMG RMS value and integrated EMG (iEMG) value and sends this information to the LCD 20 for display.

### Audio Processing

In audio processing, the differential amplifier 7, described above, sends the single ended signal to the amplfier 16 for further amplfication and then sends the signal to the band-pass filter 17 which filters out the frequencies outside of the EMG audio spectrum. The signal is then passed to the power amplfier 18, controlled by the CPU 1, which amplifies the signal based on the user adjustable volume parameter. The amplified signal is then passed to the speaker 19 for audio output.

### Stimulation Mode

In stimulation mode, the CPU 1 detects when the MODE button has been pressed to enter stimulation mode and when detected, sets the relay 14 to disconnect the electrode interface 15 from the buffer 8 and connect the electrode interface to the bridge output circuit 13. The CPU 1 sends user adjustable stimulation frequency and stimulation pulse width information to the stimulation logic circuit 9 which uses this information to control the bridge output circuit 13. The CPU 1 concurrently sends the user adjustable stimulation current parameter information to the DAC 10 which provides a variable reference voltage to the constant current circuit 12 to achieve the desired stimulus output current. The power boost 11, enabled by the CPU 1 when entering stimulation mode, provides the necessary compliance voltage of 300V to the constant current circuit 12. The constant current circuit 12 serves to detect and provide the correct current/voltage necessary to acheive the correct stimulation output based on the load seen on the bridge output circuit 13. The constant current circuit 12 then sends its output to the bridge circuit 13 which uses the constant current circuit 12 output and stimulation logic 9 output information to generate the correct stimulation current level, stimulation frequency and stimulation pulse width for output through the relay 14 and electrode interface 15 and hence to the electrodes to thereby stimulate the patient.

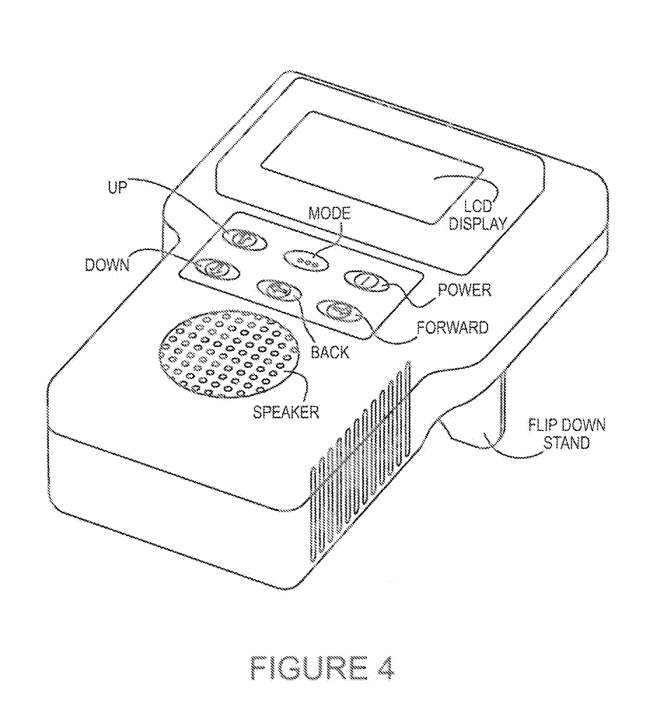

FIG. 4 is a perspective view of a non-limiting embodiment of the electromyographic (EMG) device showing the main front panel of the packaged device. In this embodiment, the main unit is packaged in a plastic enclosure that is: 5.9″ L×4.0″ W×2.1″ H (150 mm×100 mm×54 mm). The device weighs approximately 0.5 pounds (225 grams), including four AA batteries in the battery compartment. The unit is designed to be compact enough for handheld use, close to the subject patient.

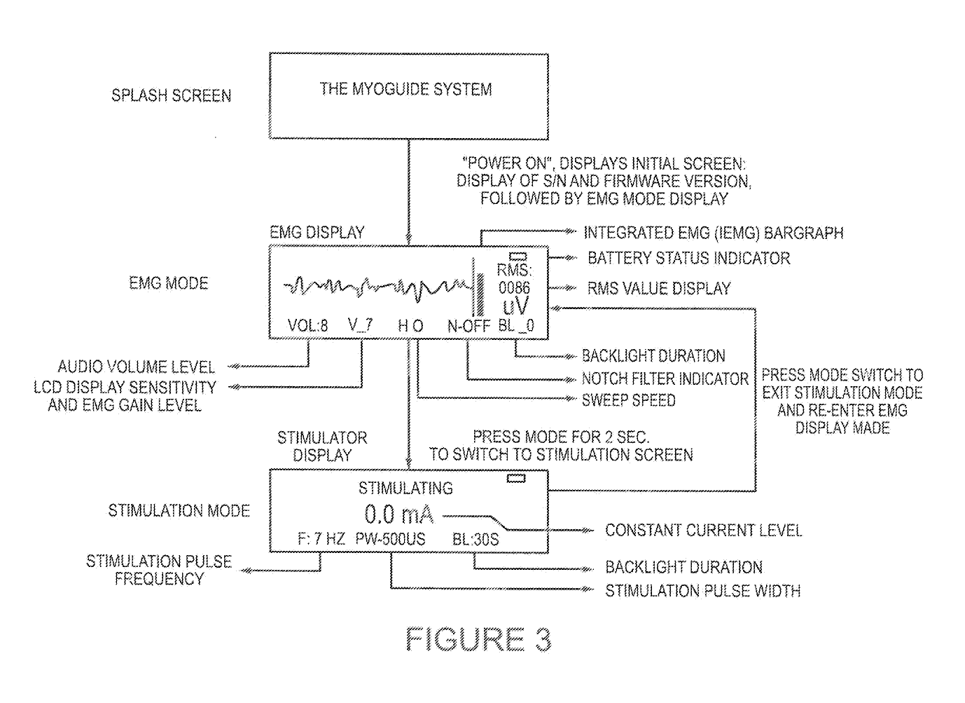

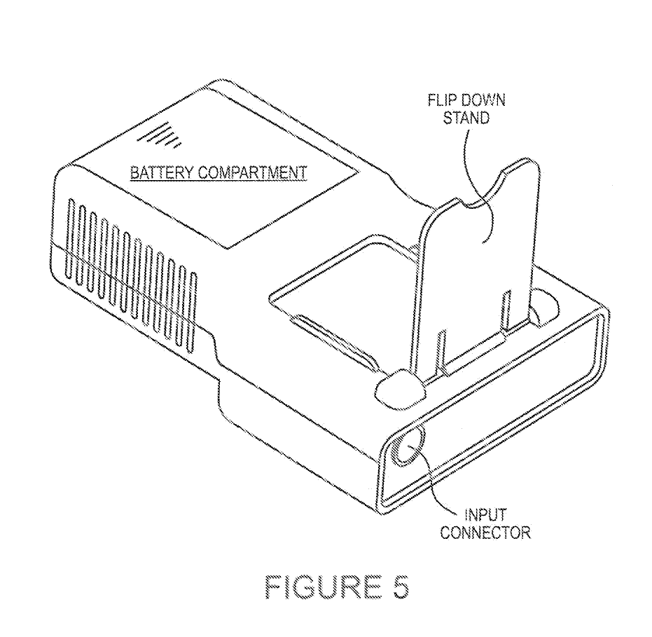

The top end panel of the device has a single touchproof, male, panel mounted, connector (see FIG. 5). This connector is designed to mate with the input cable (see FIG. 3). Referring again to FIG. 4, the front panel consists of a liquid crystal display (LCD), <\textcircled{1}> power switch, <Mode> switch for selection of EMG monitoring or stimulation, up and down (<↑> <↓>) switches for parameter level control, back and forward (<←><→>) switches to select adjustable parameters, and a speaker grill.

The LCD 20 display will indicate the system status for power, mode, stimulation level, stimulation pulse details: rate, width, LCD sensitivity and EMG gain, audio volume level, battery power status, display sweep rate and backlight time duration The LCD panel will extinguish when the unit is off.

FIG. 5 is a perspective view of the rear panel of the electromyographic device and the rear panel contains the AA battery compartment, and a flip down stand. The single input cable simplifies patient connection and reduces tangled electrode leads (see FIG. 3). The touch-proof (hypodermic) needle electrode connector allows for use of electrodes from a wide variety of different manufacturers.

The EMG device is configured to display the EMG waveform of the EMG channel on the LCD panel. The device is configured so that EMG signal display adjustment may be carried out using the LCD display sensitivity (vertical), and scan speed (horizontal) settings. The LCD display sensitivity, or vertical sensitivity, is set from the a pre-selected menu accessible on the LCD menu by the <←><→> switches. LCD display sensitivity [V] (vertical) can be set to one of 9 steps with the <↑> <↓> switches. The LCD display scanning velocity, or horizontal scan rate, is set from the [H] menu accessible on the LCD menu by the <←><→> switches. LCD display scan rate (horizontal) can be set to one of 5 steps with the <↑> <↓> switches.

FIG. 3 shows the EMG device flow diagram. The large LCD display 20 provides the complete system status at a glance. EMG audio, EMG signal display, EMG RMS (root mean square) Value, Integrated EMG signal strength and stimulation capability, increases efficacy for injection point localization. The simple control panel is intuitive and easy to operate.

| EMG Display parameter | Range | Increment |
|---|---|---|
| Audio Volume [VOL] | 1-8 | steps |
| Vertical Sensitivity [V] | 1-9 | steps |
| Horizontal Sensitivity [H] | 0, 1, 2, 4 and 8 | As selected |
| Notch Filter [N] | ON, OFF | As selected |
| Backlight [BL] | 0, 30, 60, and ON | As selected |
| | (continuous)S | |

As mention above, the EMG system operates in two modes: “[EMG]” and “[Stimulation]”. The default mode, “[EMG]”, records electromyographic (EMG) signals from electrodes A, B and C placed on the subject patient. The second mode, “[Stimulation]”, enables Myoguide's onboard stimulator to stimulate through the needle electrode A that was used to record the EMG. This enables the clinician to record and stimulate through the same needle electrode. The <Mode> switch is used to change the state of operation.

In the EMG mode, the device may be used for needle electrode examinations. Throughout the procedure, device will emit a series of audible signals from speakers 19 varying in intensity and frequency that will help in monitoring the localization of the targeted muscle or nerve. Higher levels and frequency components in the audio signal signify a higher level of EMG activity. The EMG signal may be monitored on the LCD display panel 20. A rectified and integrated EMG signal bar graph and RMS value may be displayed to monitor a gross overview of the activity.

The second mode, “Stimulation”, enables the devices onboard stimulator to stimulate through the needle electrode used to record the EMG. This enables the clinician to record and stimulate through the same needle electrode. When operated in the stimulation mode, the system may be programmed so that the LCD panel 20 displays the “Stimulating” message and the yellow indicator beside the Mode switch will illuminate when a stimulation current is being output. The stimulation status levels are displayed on the LCD panel 20: Stimulation level (mA), Pulse frequency [F] (Hz), Pulse width [PW] (uS), backlight duration [BL], and battery strength. In the stimulation mode, a current pulse train is delivered to the patient. The EMG device is preferably programmed so that the stimulation current level always defaults to 0 mA when the stimulation mode is entered.

The EMG device is configured so that in stimulation mode, the stimulation parameters are adjustable in amplitude, frequency, and pulse bandwidth. Preferred ranges for amplitude, frequency and pulse width are shown below.

| Stimulation parameter | Range | Increment |
|---|---|---|
| Amplitude | 0-20 mA 1.0 mA | steps |
| Frequency [F] | 1, 3, 5, 7, or 10 Hz | As selected |
| Pulse width [PW] | 50, 100, 200, and 500 uS | As selected |
| Backlight [BL] | 0, 30, 60, and ON | As selected |
| | (continuous)S | |

It will be appreciated that these stimulation parameters are preferred and the present invention is not restricted to any of these values. The CPU 1 of the EMG device disclosed herein is programmed to satisfy several software requirement specifications, including detection of power key press, storage and display of unit serial number and firmware revision number, the firmware of the EMG device then controls the device in two (2) modes, EMG Display Mode and Stimulation Mode, and automatic power off function to save battery power after a period of inactivity.

### EMG Mode

The CPU 1 is programmed to control and display of the EMG input signal amplification and sweep speed, control of the audio hardware to provide volume control function and mute function, ON/Off control of hardware notch filter, control of and display LCD display backlight duration, calculation and display of input signal RMS value, calculation and bar graph display of integrated EMG value, key press detection to allow user to change above parameters, battery status display and low battery indication function, and mode switch key press detection to allow change to Stimulation mode.

### Stimulation Mode

In stimulation mode, the CPU 1 is programmed, for safety purposes, so that the stimulation current output always defaults to 0.0 mA when entering Stimulation mode, for safety purposes, extended duration key press of Mode switch required to enter Stimulation mode, display “Stimulating” when in Stimulation Mode, control of the stimulation output current, stimulation frequency, stimulation pulse width and display of output current setting, control of stimulation LED to turn LED on when stimulation is being delivered, key press detection to allow stimulation to be turned on or off (paused), display of “Stimulation Paused” when stimulation has been paused, storage and retrieval of user parameters when entering/exiting stimulation pause function, control of and display of LCD display backlight duration, key press detection to allow user to change above parameters, battery status display and low battery indication function, mode switch key press detection to allow change to EMG Display mode, storage and retrieval of user parameter settings except for output current when entering/exiting Stimulation mode.

The firmware upon detection of power key press displays a splash screen showing device name, device serial number and firmware revision number. The firmware then enters EMG Display mode.

In EMG Display mode, the firmware displays input EMG waveform, integrated EMG bar graph, input EMG RMS value and current audio volume (VOL: parameter), vertical sensitivity (amplification) (V: parameter), horizontal sweep speed (H: parameter), notch filter (N: parameter) and backlight parameter (BL: parameter) settings. Battery status indicator is also displayed in EMG Display mode. Key press detection of control panel arrow keys allows the user to change parameter settings. Initiation of audio mute function is performed upon detection of key press combination. Key press detection of control panel Mode key initiates entering of Stimulation mode. For safety purposes and extended Mode key press is required to enter Stimulation mode. Storage of EMG Display mode parameters is performed when exiting EMG display mode to allow for retrieval of user settings when re-entering EMG Display mode.

In Stimulation mode, the firmware always defaults the stimulation output current to 0.0 mA when entering Stimulation mode for safety purposes. Stimulation screen is displayed showing current parameter settings for stimulation output current, stimulation frequency (F: parameter), stimulation pulse width (PW: parameter) and backlight duration (BL: parameter). Battery status indicator is also displayed. Key press detection of control panel arrow keys allows the user to change parameter settings. When stimulation output current is parameter is increased, the firmware controls the stimulation output current, frequency and pulse width based on the current parameter settings and the stimulation LED is turned ON. Stimulation pause function is initiated by detection of key press combination and stimulation output and stimulation LED is turned off. Storage of all stimulation parameter settings is performed so that they can be retrieved when pause function is exited. Detection of control panel button key press combination resumes stimulation. Mode switch key press detection allows switching back to EMG Display Mode.

Battery low warning indication is provided in both EMG Display and Stimulation modes to alert the user that batteries need to be replaced. Automatic power off function turns unit off after a duration of inactivity to increase battery life.

The central process controller 30 is programmed with fully upgradable firmware. The firmware is upgradeable via a programming port located on the pcb inside the enclosure. PCB need not be removed for firmware upgrade. Firmware upgrade accomplished using serial cable and software running on a host computer.

The present disclosure describes a method of identifying trigger points in muscle associated with a characteristic spontaneous EMG activity, and/or Stimulation location procedures. The trigger point locations are found by physical examination, and manual palpation. They are found to be nodular in nature, or “tight spots” in the muscle. These areas are marked for easy location, once the needle EMG guided injection procedure is ready. It is quite difficult to find the optimal injection locations without the EMG device disclosed herein, as there is a subtlety related to the depths where hyperactive muscle fibers reside. The present EMG device allows the clinician to see (integrated visual display) and hear (EMG audio output) the EMG signal, providing confirmation that the needle electrode A is indeed in the best location. Another important aspect relates to re-injected sites. Sites injected in previous encounters may still exhibit a degree of neuromodulation. It is of benefit to be able to see the EMG signal, as it provides information about activity that EMG audio alone cannot. The injection dose can be titrated to suit the activity. Hence, we now have a way to evaluate where the optimal injection site is and how much medication is appropriate to inject. This helps to prevent injection of higher volumes and can reduce the concomitant diffusion to adjacent sites.

In addition to the value of the present EMG device in finding the injection site, as indicated by a significantly increased level of EMG activity, several layers of different activity can be found that may lead to multiple injections along the same injection tract. This cannot be done without such a device.

Multi-focal sites, such as the fingers (or toes), can require the use of stimulation to identify the correct injection sites. EMG itself cannot separate the different fingers (or toes), as they all emit EMG signals. Stimulation will invoke a twitch response and identify the finger.

A significant advantage of the EMG device disclosed herein comes from its integrated stimulation capability. Stimulation pulses can be specified, and delivered through the needle electrode A to aid in finding the injection site, as indicated by the twitch response. Stimulation, or E-stim, location is essential in these areas.

Stimulation parameters can be changed easily with several pulse widths, frequency of pulse presentation, and constant current level. The EMG device is configured so that a bright yellow light is illuminated when actively stimulating, as per IEC 60601. Thus the EMG device disclosed herein is a very advantageous tool, whenever drugs are injected into muscle, by virtue of its ability to provide visual and auditory feedback related to the EMG at the injection site.

The present EMG device provides a superior injection site targeting system with many useful features, such as, the ability to see and hear EMG signals, display real time analyzed EMG, and stimulation location capability. There are numerous advantages associated with the present EMG device for EMG guidance. These advantages include the fact that the device is conveniently integrated into one handheld package. It helps identify involved muscles i.e. pre-injection physiopathological evaluation, or pre-intervention evaluation. (Either by EMG or stimulation location). The device is useful for pre-injection evaluation in cases where the site may be surrounded by essential nerves and blood vessels. Pre-injection evaluation can lead to reduced drug dose and volume, thereby reducing the incidence of drug resistance, and limiting drug diffusion into adjacent areas.

In summary, the present invention provides an electromyographic (EMG) device for diagnosing chronic and recurrent muscle pain which includes to two active EMG electrodes and an EMG reference electrode which when attached to the subject patient can measure and display electromyographic signals. The device includes an integrated stimulator can be used for treating chronic and recurrent muscle pain, by evoking stimulation, or E-stim, location. The device includes a visual display system for displaying raw and processed EMG on an easy to read LCD display, allowing visual analysis of the level of spontaneous EMG activity in trigger points and adjacent muscle tissue.

The device is configured to provide for propagation of audio feedback based on the raw EMG. This provides an auditory analysis of the level of spontaneous EMG activity in trigger points and adjacent muscle tissue.

The need to increase accuracy when injecting neuromodulators has been highlighted by recent FDA reviews of the dangers of peripheral effects. Use of the present device can lead to more accurate injection site targeting, allowing reduced concentrations and volumes of injected drugs, thus the EMG device can help increase treatment efficacy.

Some aspects of the present disclosure can be embodied, at least in part, in software. That is, the techniques can be carried out in a computer system or other data processing system in response to its processor, such as a microprocessor, executing sequences of instructions contained in a memory, such as ROM, volatile RAM, non-volatile memory, cache, magnetic and optical disks, or a remote storage device. Further, the instructions can be downloaded into a computing device over a data network in a form of compiled and linked version. Alternatively, the logic to perform the processes as discussed above could be implemented in additional computer and/or machine readable media, such as discrete hardware components as large-scale integrated circuits (LSI's), application-specific integrated circuits (ASIC's), or firmware such as electrically erasable programmable read-only memory (EEPROM's).

Central processor controller unit (CPU) 1 shown in FIG. 1 provides an example implementation of control and processing unit, which may include one or more processors (for example, a CPU/microprocessor), a bus, memory which may include random access memory (RAM) and/or read only memory (ROM), one or more internal storage devices (e.g. a hard disk drive, compact disk drive or internal flash memory), a power supply, one more communications interfaces, external storage, a display and various input/output devices and/or interfaces (e.g., a receiver, a transmitter, a speaker, a display, an imaging sensor, such as those used in a digital still camera or digital video camera, a clock, an output port, a user input device, such as a keyboard, a keypad, a mouse, a position tracked stylus, a position tracked probe, a foot switch, and/or a microphone for capturing speech commands).

In one embodiment, control and processing unit (CPU) 1 may be, or include, a general purpose computer or any other hardware equivalents. Control and processing unit 1 may be implemented using application specific integrated circuits (ASIC). Alternatively, control and processing unit 1 can be implemented as a combination of hardware and software, where the software is loaded into the processor from the memory or over a network connection.

Control and processing unit 1 may be programmed with a set of instructions which when executed in the processor causes the system to perform one or more methods described in the disclosure. Control and processing unit 1 may include many more or less components than those shown.

While some embodiments have been described in the context of fully functioning computers and computer systems, those skilled in the art will appreciate that various embodiments are capable of being distributed as a program product in a variety of forms and are capable of being applied regardless of the particular type of machine or computer readable media used to actually effect the distribution.

A computer readable medium can be used to store software and data which when executed by a data processing system causes the system to perform various methods. The executable software and data can be stored in various places including for example ROM, volatile RAM, non-volatile memory and/or cache. Portions of this software and/or data can be stored in any one of these storage devices. In general, a machine readable medium includes any mechanism that provides (i.e., stores and/or transmits) information in a form accessible by a machine (e.g., a computer, network device, personal digital assistant, manufacturing tool, any device with a set of one or more processors, etc.).

Examples of computer-readable media include but are not limited to recordable and non-recordable type media such as volatile and non-volatile memory devices, read only memory (ROM), random access memory (RAM), flash memory devices, floppy and other removable disks, magnetic disk storage media, optical storage media (e.g., compact discs (CDs), digital versatile disks (DVDs), etc.), among others. The instructions can be embodied in digital and analog communication links for electrical, optical, acoustical or other forms of propagated signals, such as carrier waves, infrared signals, digital signals, and the like.

As used herein, the terms, “comprises” and “comprising” are to be construed as being inclusive and open ended, and not exclusive. Specifically, when used in this specification including claims, the terms, “comprises” and “comprising” and variations thereof mean the specified features, steps or components are included. These terms are not to be interpreted to exclude the presence of other features, steps or components.

The foregoing description of the preferred embodiments of the invention has been presented to illustrate the principles of the invention and not to limit the invention to the particular embodiment illustrated. It is intended that the scope of the invention be defined by all of the embodiments encompassed within the following claims and their equivalents.

Therefore what is claimed is:

1. A electromyographic (EMG) device, comprising:
a) a central processor controller and a user interface, an audio processing circuit connected to at least one speaker, an array of electrodes having first ends configured to be affixed to the skin of a subject patient, an electrode interface, said array of electrodes having second ends connected to said electrode interface, at least one of said electrodes is a hypodermic needle electrode;
b) an acquisition subsystem for measuring and recording electromyographic (EMG) activity connected to the central processor controller, the acquisition subsystem including
i) electromyographic signal detection and processing circuits connected to said electrode interface and said central processor controller for measuring electromyographic signals picked up by said array of electrodes and simultaneously transmitting said electromyographic signals to said audio processing circuit and processing said electromyographic signals to put them in a selected format and transmitting the formatted signals to said central processor controller;
c) a stimulation subsystem connected to the central processor controller, the stimulation subsystem including
i) stimulation logic circuit to control parameters of stimulation signals applied to the subject patient, said parameters including timing of stimulation signal frequency, stimulation signal pulse width, and stimulation signal pulse amplitude, the stimulation logic circuit being connected to the central processor controller,
ii) an electrical signal generator circuit connected to said central processor controller,
iii) a bridge output circuit connected to said stimulation logic circuit and said electrical signal generator circuit configured to produce said stimulation signals for application to the subject patient based on input from the stimulation logic circuit and the electrical signal generator circuit, the bridge circuit being connected to said electrode interface for delivering said stimulation signals to the subject patient through the array of electrodes;
d) a switch for switching between said acquisition subsystem and said stimulation subsystem; and
e) a visual display connect to said central processor controller configured to display to a clinician at least real-time electromyographic waveforms from said acquisition subsystem and stimulation parameters from said stimulation subsystem.

2. The electromyographic device according to claim 1 wherein said electrical signal generator circuit is a constant current generator circuit, and wherein said stimulation subsystem includes a digital-to-analog converter connected to said central processor controller and said constant current generator circuit to provide a variable reference voltage to the constant current circuit.

3. The electromyographic device according to claim 2 wherein said stimulation logic circuit is configured to control stimulation parameters such as current intensity, pulse width, and frequency.

4. The electromyographic device according to claim 1 wherein said electromyographic signal detection and processing circuits include a buffer circuit connected to said electrode interface, including a differential amplifier, wherein said buffer circuit is configured to increase input impedance from said electrode interface.

5. The electromyographic device according to claim 4 wherein said switch for switching between said acquisition subsystem and said stimulation subsystem is a relay circuit connected between said bridge output circuit and said electrode interface and connected between said buffer circuit and said electrode interface.

6. The electromyographic device according to claim 4 wherein said electromyographic signal detection and processing circuits include frequency trap circuit connected between a first output of said differential amplifier and an analog switch, the differential amplifier being connected directly to said analog switch, said analog switch being connected to a band-pass filter with said band-pass filter being connected to a programmable magnifier, and said programmable magnifier being connected to said central processor controller, and wherein said frequency trap circuit is used to filter out frequency noise, said analog switch is to toggle usage of said frequency trap circuit, said band-pass filter is used to filter out noise outside an electromyographic spectrum, said programmable magnifier connected to said analog-to-digital converter is configured to enable switching to achieve different levels of magnification, of the filtered electromyographic signals, and wherein said analog-to-digital converter connected to said central processor unit and said programmable magnifier is for converting said filtered electromyographic signals to digital signals which are input to said central processor controller.

7. The electromyographic device according to claim 4 wherein a second output of said differential amplifier is connected to said audio processing circuit, said audio processing circuit including an amplifier having an input connected to said second output of said differential amplifier for performing further amplification for the electromyographic signals received from said differential amplifier, including a second band-pass filter connected to said amplifier to filter out noise outside the electromyographic spectrum on the signals output from amplifier, and said at least one speaker being connected to said second band-pass filter used to convert electrical signals into audio signals.

8. The electromyographic device according to claim 1 wherein said central processor controller is programmed to mute said speaker by command through said user interface by a clinician operating the device.

9. The electromyographic device according to claim 1 packaged in a handheld, battery operated enclosure.

10. The electromyographic device according to claim 1 wherein said visual display is configured to display raw EMG signals, an integrated EMG bar graph, and root mean square EMG indication.

11. The electromyographic device according to claim 1 wherein said visual display is a liquid crystal display.

12. The electromyographic device according to claim 1 wherein said central process controller is programmed with fully upgradable firmware.

13. The electromyographic device according to claim 3 wherein said electromyographic signal detection and processing circuits include a buffer circuit connected to said electrode interface, including a differential amplifier, wherein said buffer circuit is configured to increase input impedance from said electrode interface.

14. The electromyographic device according to claim 5 wherein said electromyographic signal detection and processing circuits include frequency trap circuit connected between a first output of said differential amplifier and an analog switch, the differential amplifier being connected directly to said analog switch, said analog switch being connected to a band-pass filter with said band-pass filter being connected to a programmable magnifier, and said programmable magnifier being connected to said central processor controller, and wherein said frequency trap circuit is used to filter out frequency noise, said analog switch is to toggle usage of said frequency trap circuit, said band-pass filter is used to filter out noise outside an electromyographic spectrum, said programmable magnifier connected to said analog-to-digital converter is configured to enable switching to achieve different levels of magnification, of the filtered electromyographic signals, and wherein said analog-to-digital converter connected to said central processor unit and said programmable magnifier is for converting said filtered electromyographic signals to digital signals which are input to said central processor controller.

15. The electromyographic device according to claim 5 wherein a second output of said differential amplifier is connected to said audio processing circuit, said audio processing circuit including an amplifier having an input connected to said second output of said differential amplifier for performing further amplification for the electromyographic signals received from said differential amplifier, including a second band-pass filter connected to said amplifier to filter out noise outside the electromyographic spectrum on the signals output from amplifier, and said at least one speaker being connected to said second band-pass filter used to convert electrical signals into audio signals.

16. The electromyographic device according to claim 6 wherein a second output of said differential amplifier is connected to said audio processing circuit, said audio processing circuit including an amplifier having an input connected to said second output of said differential amplifier for performing further amplification for the electromyographic signals received from said differential amplifier, including a second band-pass filter connected to said amplifier to filter out noise outside the electromyographic spectrum on the signals output from amplifier, and said at least one speaker being connected to said second band-pass filter used to convert electrical signals into audio signals.

17. The electromyographic device according to claim 2 wherein said central processor controller is programmed to mute said speaker by command through said user interface by a clinician operating the device.
